(19) United States
(12) Patent Application Publication
(10) Pub. No.: US 2005/0255157 A1
(43) Pub. Date: Nov. 17, 2005

(54) Sustained release, mucoadhesive vaginal pharmaceutical compositions

(75) Inventors: Nilendu Sen, Navi Mumbai (IN); Kaliaperumal Arun Prasath, Navi Mumbai (IN); Shrikant Bhonsle, Thane (IN); Anandi Krishnan, Navi Mumbai (IN)

Correspondence Address: DILWORTH & BARRESE, LLP, 333 EARLE OVINGTON BLVD., UNIONDALE, NY, 11553, US

(73) Assignee: Glenmark Pharmaceuticals Limited, Mumbai (IN)

(21) Appl. No.: 11/126,972

(22) Filed: May 11, 2005

Related U.S. Application Data
(60) Provisional application No. 60/569,865, filed on May 11, 2004.

(51) Int. Cl. A61K 31/496; A61K 9/22

(52) U.S. Cl. 424/468; 514/254.07

(57) ABSTRACT

A sustained release, mucoadhesive vaginal pharmaceutical composition is provided comprising (a) an effective amount of at least one active pharmaceutical ingredient and (b) a hydrophilic matrix having mucoadhesive properties and capable of providing a sustained release of the active pharmaceutical ingredient, the hydrophilic matrix comprising a hydrophilic polymer having a weight average molecular weight of at least about 100,000. Also provided are solid oral dosage forms comprising the sustained release, mucoadhesive vaginal pharmaceutical compositions.

### CROSS-REFERENCE TO RELATED APPLICATIONS

This application claims the benefit under 35 U.S.C. §119 to Provisional Application No. 60/569,865, filed May 11, 2004 and entitled “SUSTAINED RELEASE, MUCOADHESIVE VAGINAL PHARMACEUTICAL COMPOSITIONS”, the contents of which are incorporated by reference herein.

### BACKGROUND OF THE INVENTION

1. Technical Field

The present invention relates generally to sustained release, mucoadhesive vaginal pharmaceutical compositions suitable for use in the vaginal cavity and processes for their preparation. More specifically, the present invention is directed to sustained release, mucoadhesive vaginal pharmaceutical compositions which provide for sustained delivery of one or more active pharmaceutical ingredient for the treatment of vaginal conditions.

2. Description of the Related Art

An important therapeutic area in feminine health is the medicinal treatment of the female reproductive system. Related therapies typically involve the delivery of an API to the vaginal cavity and its environs. Generally, the vaginal cavity exhibits an aqueous environment containing secreting glands whose fluids create an acidic pH in the range of about 4-5. In its normal state, the lining of the vagina secretes a fluid that is fermented to an acid by bacteria that are normally present. About 10% of non-pregnant woman aged 15-55 harbour the yeast Candida albicans in the vagina but most have no symptoms and it is harmless to them. This acidity is a protective mechanism that helps to protect the vagina from invasion by other organisms.

Vaginal Candidiasis is a yeastlike fungi infection of the vulva and/or vagina. It causes a foul smelling, sticky, white-yellow discharge that may be accompanied by itching, burning and swelling. Such an infection can also make walking, urinating, or intercourse painful.

Certain drugs therapies can alter the balance of natural organisms that are present in the vagina, and hereby promote the growth of Candida. Examples include the extended use of antibiotics, steroids and oral contraceptives with high estrogen content. Other factors which may cause candidiasis include, diabetes, pregnancy, using antihistamines, iron, folate, vitamin B12, or zinc deficiency. Factors that may weaken the immune system (from cancer therapy to stress and depression) can also cause candidiasis. Tight fitting pants and the reactions to chemical ingredients found in soaps and detergents may also lead to vaginal candidiasis.

Due to the physiological functions and conditions described above, the vaginal cavity is susceptible to disease and infection. In attempting to treat such conditions, it has proven difficult to deliver an API to this area in a sustained manner for an extended period of time.

Treatment with topical antifungal compositions, such as creams or suppositories, are normally the first choice of treatment for mild to moderate yeast infections. Serious infections, however, require a longer course of treatment (7-14 days). These dosage forms are inconvenient to use and with longer courses of therapy may result in patient noncompliance.

In general, the above topical treatments have moderate side effect profiles. Some subjects, however, experience more complicated side effects, including vaginal burning, itching, or skin rash. In addition, some patients experience cramps or headache.

Currently available treatment of vaginal candidiasis includes topical administration using vaginal creams, ointments or vaginal suppositories, which release the drug from these dosage forms by melting or dissolving in the vagina. Such dosage forms ultimately get discharged from the vagina resulting in unsanitary conditions, discomfort and unpredictable delivery of the active drug.

Accordingly, there remains a need for a SR vaginal pharmaceutical composition with mucoadhesive properties to provide for a sustained delivery of an active pharmaceutical ingredient (API) to the vagina for the treatment of, for example, vaginal candidiasis.

### SUMMARY OF THE INVENTION

One aspect of the present invention is to a sustained release mucoadhesive, vaginal pharmaceutical composition which provides sustained release of at least one active pharmaceutical ingredient (API) to the site of absorption or action in the vagina, e.g., for at least about 3 to about 12 hours. Another aspect of the present invention provides a mucoadhesive, vaginal delivery dosage system which releases an antifungal agent to the site of absorption or action in a sustained manner, e.g., for at least about 3 to about 12 hours, in the vagina of a human female.

Yet another aspect of the present invention provides for processes of preparing vaginal delivery dosage systems which release an effective amount of at least one active pharmaceutical ingredient to the site of absorption or action in a sustained manner. The delivery systems of the present invention are bioadherent to the epithelial tissues in the vagina.

Accordingly, in one embodiment of the present invention, a sustained release, mucoadhesive vaginal pharmaceutical composition is provided comprising an effective amount of at least one active pharmaceutical ingredient and a hydrophilic matrix having mucoadhesive properties and capable of providing a sustained release of the active pharmaceutical ingredient, the hydrophilic matrix comprising a hydrophilic polymer having a weight average molecular weight of at least about 100,000.

In accordance with a second embodiment of the present invention, a method for treating a vaginal condition in a human female is provided comprising the step of administering into the vaginal cavity of the human female a sustained release, mucoadhesive vaginal pharmaceutical composition comprising an effective amount of at least one active pharmaceutical ingredient and a hydrophilic matrix having mucoadhesive properties and capable of providing a sustained release of the active pharmaceutical ingredient, the hydrophilic matrix comprising a hydrophilic polymer having a weight average molecular weight of at least about 100,000.

In accordance with another embodiment of the present invention, processes for preparing a sustained release, mucoadhesive, vaginal composition are provided utilizing, for example, wet granulation, dry granulation, slugging, roll compaction, and direct compression.

In accordance with yet another embodiment of the present invention, a kit for delivering the sustained release mucoadhesive, vaginal composition to the site of absorption or action in the vaginal cavity of a human female is provided which includes one or more of the solid oral sustained release, mucoadhesive, vaginal compositions of the present invention and an applicator suitable for delivering the composition to the site in the vaginal cavity. Such kits can also include, for example, other compounds and/or compositions (e.g., permeation enhancers, lubricants), and written instructions in a form prescribed by a governmental agency regulating the manufacture, use or sale of pharmaceuticals or biological products, which instructions can also reflects approval by the agency of manufacture, use or sale for human administration. For example, the kit may be used in a hospital setting for administering the active pharmaceutical ingredient, e.g., oxitocin or petocin, by way of the applicator to a human female in the delivery room to induce labor.

The present invention provides several advantages over the prior art, including:
1. Providing a continuous concentration of an API to the vaginal epithelium and the mucosa. The present invention prevents discharge of the API out of the vagina through the bioadhesive properties of the composition to maintain continuous contact with the vaginal mucosa.
2. Improved side effect profile and patient compliance.
3. Formation of a semisolid gel like soft mass having bioadherent properties on contact with biogical fluid from which the API is released in a sustained manner.
   Definitions

The term “treating” or “treatment” of a state, disorder or condition as used herein means: (1) preventing or delaying the appearance of clinical symptoms of the state, disorder or condition developing in a mammal that may be afflicted with or predisposed to the state, disorder or condition but does not yet experience or display clinical or subclinical symptoms of the state, disorder or condition, (2) inhibiting the state, disorder or condition, i.e., arresting or reducing the development of the disease or at least one clinical or subclinical symptom thereof, or (3) relieving the disease, i.e., causing regression of the state, disorder or condition or at least one of its clinical or subclinical symptoms. The benefit to a subject to be treated is either statistically significant or at least perceptible to the patient or to the physician.

The term “effective amount” as used herein means therapeutic amount of a compound that, when administered to a mammal for treating a state, disorder, condition or causing an action, e.g., inducement of female into labor, is sufficient to effect such treatment or action. The “effective amount” will vary depending on the compound, the condition to treated and its severity and the age, weight, physical condition and responsiveness of the mammal to be treated.

The term “delivering” as used herein means providing a therapeutically effective amount of an active ingredient to a particular location within a host means causing a therapeutically effective blood concentration of the active ingredient at the particular location. This can be accomplished, e.g., by topical, local or by systemic administration of the active ingredient to the host.

By “pharmaceutically acceptable” is meant those salts and esters which are, within the scope of sound medical judgment, suitable for use in contact with the tissues of humans and lower animals without undue toxicity, irritation, allergic response and the like, commensurate with a reasonable benefit/risk ratio, and effective for their intended use. Representative acid additions salts include the hydrochloride, hydrobromide, sulphate, bisulphate, acetate, oxalate, valerate, oleate, palmitate, stearate, laurate, borate, benzoate, lactate, phosphate, tosylate, mesylate, citrate, maleate, fumarare, succinate, tartrate, ascorbate, glucoheptonate, lactobionate, lauryl sulphate salts and the like. Representative alkali or alkaline earth metal salts include the sodium, calcium, potassium and magnesium salts, and the like.

The term “subject” or “a patient” or “a host” as used herein refers to mammalian animals, preferably human.

As used herein, the term “antioxidant” is intended to mean an agent who inhibits oxidation and is thus used to prevent the deterioration of preparations by the oxidative process. Such compounds include, by way of example and without limitation, ascorbic acid, ascorbic palmitate, Vitamin E, butylated hydroxyanisole, butylated hydroxytoluene, hypophosphorous acid, monothioglycerol, propyl gallate, sodium ascorbate, sodium bisulfite, sodium formaldehyde sulfoxylate, sodium metalbisulfite and other such materials known to those of ordinary skill in the art.

As used herein, the term “buffering agent” is intended to mean a compound used to resist a change in pH upon dilution or addition of acid of alkali. Such compounds include, by way of example and without limitation, potassium metaphosphate, potassium phosphate, monobasic sodium acetate and sodium citrate anhydrous and dehydrate and other such material known to those of ordinary skill in the art.

As used herein, the term “binders” is intended to mean substances used to cause adhesion of powder particles in tablet granulations. Such compounds include, by way of example and without limitation, acacia alginic acid, tragacanth, carboxymethylcellulose sodium, poly(vinylpyrrolidone), compressible sugar (e.g., NuTab), ethylcellulose, gelatin, liquid glucose, methylcellulose, povidone and pregelatinized starch, combinations thereof and other material known to those of ordinary skill in the art.

When needed, other binders may also be included in the present invention. Exemplary binders include starch, poly(ethylene glycol), guar gum, polysaccharide, bentonites, sugars, invert sugars, poloxamers (PLURONIC™ F68, PLURONIC™ fl27), collagen, albumin, celluloses in nonaqueous solvents, combinations thereof and the like. Other binders include, for example, poly(propylene glycol), polyoxyethylene-polypropylene copolymer, polyethylene ester, polyethylene sorbitan ester, poly(ethylene oxide), microcrystalline cellulose, poly(vinylpyrrolidone), combinations thereof and other such materials known to those of ordinary skill in the art.

As used herein, the term “wetting agent” is intended to mean a compound used to aid in attaining intimate contact between solid particles and liquids. Exemplary wetting agents include, by way of example and without limitation, gelatin, casein, lecithin (phosphatides), gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters, (e.g., TWEEN™s), polyethylene glycols, polyoxyethylene stearates colloidal silicon dioxide, phosphates, sodium dodecylsulfate, carboxymethylcellulose calcium, carboxymethylcellulose sodium, methylcellulose, hydroxyethylcellulose, hydroxylpropylcellulose, hydroxypropylmethylcellulose phthalate, noncrystalline cellulose, magnesium aluminum silicate, triethanolamine, polyvinyl alcohol, and polyvinylpyrrolidone (PVP). Tyloxapol (a nonionic liquid polymer of the alkyl aryl polyether alcohol type, also known as superinone or triton) is another useful wetting agent, combinations thereof and other such materials known to those of ordinary skill in the art.

Most of these excipients are described in detail in, e.g., Howard C. Ansel et al., Pharmaceutical Dosage Forms and Drug Delivery Systems, (7th Ed. 1999); Alfonso R. Gennaro et al., Remington: The Science and Practice of Pharmacy, (20th Ed. 2000); and A. Kibbe, Handbook of Pharmaceutical Excipients, (3rd Ed. 2000), the contents of which are incorporated by reference herein.

### BRIEF DESCRIPTION OF THE DRAWINGS

FIG. 1 is a top plan view of a vaginal tablet in accordance with an embodiment of the present invention;

FIG. 2 is a front elevational view in the direction of arrow 2 of the vaginal tablet shown in FIG. 1;

FIG. 3 is a left side elevational view of the vaginal tablet shown in FIGS. 1 and 2; and

FIG. 4 is a bottom plan view of the vaginal tablet shown in FIGS. 1-3.

### DETAILED DESCRIPTION OF THE PREFERRED EMBODIMENTS

The present invention provides for a sustained release, mucoadhesive vaginal pharmaceutical composition comprising (a) an effective amount of at least one active pharmaceutical ingredient and (b) a hydrophilic matrix having mucoadhesive properties and capable of providing a sustained release of the active pharmaceutical ingredient, the hydrophilic matrix comprising a hydrophilic polymer having a weight average molecular weight of at least about 100,000. In one embodiment, the sustained release, mucoadhesive vaginal pharmaceutical composition is a solid oral dosage form, e.g., a tablet, capsule, etc.

The sustained release, mucoadhesive vaginal pharmaceutical compositions of the present invention are useful in sustaining delivery of one or more active pharmaceutical ingredients in the vaginal cavity of a human female subject for the treatment of vaginal conditions such as, for example, specific and non specific vaginal infections (due to, e.g., bacteria, fungi, and protozoa); cervical and vulval infections; prevention of recurrent infection of vagina and lower urinary tract; hormonal disorders, e.g., endometriosis, leucorrhoea, and postmenopausal vaginitis; cervical priming; induction and augmentation of labor; supplementation of luteal phase in case of sterility due to luteal deficiency; treatment of threatened abortions; dyspareunia after menopause and vaginal surgery; puerperal depression and to help pregnancy, e.g., inducement of labor, and in contraception.

Accordingly, the active pharmaceutical ingredients for use in the vaginal pharmaceutical compositions of the present invention include, but are not limited to, antifungal agents, prostaglandins, hormones, estrogens, pharmaceutically acceptable salts or esters thereof, isomers, derivatives thereof and the like and combinations thereof.

By way of alleviating a fungal infection in the vaginal cavity, any of the antifungal agents heretofore used to alleviate a fungal infection in the vaginal cavity can be used herein. Suitable antifungal agents for use in the pharmaceutical compositions herein include those disclosed in, for example, Remington's Pharmaceutical Sciences, 16th Ed., Mack Publishing Co., Easton, Pa. Pp. 1225-1231 (1980) and in Goodman and Gilman's The Pharmacological Basis of Therapeutics by Hardman and Limbird, 9th Ed., McGraw-Hill, N.Y, pp. 1165-1181 (1996), the contents of which are incorporated by reference herein. Suitable antifungal agents for use herein includes any azole-containing antifungal agent known to one skilled in the art including, by way of example, imidazoles, triazoles, pharmaceutically acceptable salts or esters thereof, isomers, derivatives thereof, and the like and mixtures thereof. Useful imidazoles include but are not limited to, econazole, clotrimazole, metronidazole, tioconazole, fenticonazole, isoconazole, ketoconazole, sulconazole, bifonazole, omoconazole, azanidazole, butoconazole, oxiconazole, pharmaceutically acceptable salts or esters thereof, isomers, derivatives thereof, and the like and mixtures thereof. Useful triazoles include, but are not limited to, fluconazole, terconazole, itraconazole pharmaceutically acceptable salts or esters thereof, isomers, derivatives thereof, and the like and mixtures thereof. Preferably, the azole-containing antifungal agent is clotrimazole. Clotrimazole is a broad-spectrum, anti-fungal agent that is used for the treatment of dermal infections caused by, for example, various species of pathogenic dermatophytes, yeasts, and malassezia furtur. In vitro, clotrimazole exhibits fungistatic and fungicidal activity against isolates of, for example, Trichophyton rubrum, Trichophyton mentagrophytes, Epidermophyton floccosum, Microsporum canis, Candida spp., and Malassezia pachydermatis (Pityrosporum canis).

Suitable prostaglandins, hormones, and estrogens for use in the pharmaceutical formulation herein include those disclosed in Remington's Pharmaceutical Sciences, 16th Ed., 1980, Mack Publishing Co., Easton, Pa. and in Goodman and Gilman's The Pharmacological Basis of Therapeutics by Hardman and Limbird, 9th Ed., 1996, McGraw-Hill, N.Y, the contents of which are incorporated by reference herein. Examples of prostaglandins include, but are not limited to, arbaprostil, carboprost, cloprostenol, dinoprost, dinoprostone, doxaprost, epoprostenol sodium, fenprostalene, fluprostenol, gemeeprost, meteneprost, prostalene, petocin, misoprostol, sulprostone, tiaprost, pharmaceutically acceptable salts or esters thereof, isomers, derivatives thereof, and the like and mixtures thereof.

The hydrophilic matrix for use in the sustained release, mucoadhesive vaginal pharmaceutical composition of the present invention advantageously provides mucoadhesive properties and a sustained release of the active pharmaceutical ingredients when present in the vaginal cavity. For example, the hydrophilic matrix used herein may release the active ingredients over a time period ranging from about 3 to about 12 hours, preferably about 4 to about 8 hours and more preferably about 4 to about 6 hours time. It may also exhibit the mucoadhesion properties after insertion of the compositions of this invention into the vaginal cavity within about 60 second time to hold the composition intact at the desire site of absorption or action to release the active pharmaceutical ingredients for treatment in a sustained manner. The hydrophilic matrix generally includes water soluble, hydrophilic, high molecular weight, polymers having hydrogen bonding functionality and good biocompatibility. The adhesion properties of these polymers are the result of the entanglement of polymer chains and interactions with glycoproteins of the mucosal surface. The chemical nature of the bioadhesive polymers, including chain and side groups and crosslinking agents, generates interactions between the mucosal constituents and the polymer or polymers, such as physical entanglement, Van der Waals interactions, and hydrogen bonding. Thus the long linear chain structure allows them to form a strong interpenetrating network with mucus and help in effective bioadhesion. Accordingly, the hydrophilic matrix of the present invention includes at least one or more hydrophilic polymers having a weight average molecular weight of at least about 100,000, preferably a weight average molecular weight of at least about 500,000, more preferably a weight average molecular weight ranging from about 1,000,000 to about 10,000,000 and most preferably a weight average molecular weight of from about 2,000,000 to about 6,000,000.

Suitable hydrophilic polymers include, but are not limited to, polyalkylene oxides such as C₁-C₁₀ polyalkylene oxides and preferably C₂-C₁₀ polyalkylene oxides with polyethylene oxides being most preferred. The polyethylene oxides are generally water soluble, nonionic homopolymers of ethylene oxide, which represents the average number of oxyethylene groups (about 2000 to over about 200,000). In the present invention the higher molecular weight grades advantageously provide sustained drug release. Upon exposure to water or gastric juices, the polyalkylene oxides hydrate and swells rapidly to form hydrogels with properties ideally suited for controlled drug delivery.

In one embodiment of the present invention, a polyethylene oxide (e.g., POLYOX of DOW) having a weight average molecular weight of about 2,000,000 to about 5,000,000 with a viscosity at 25° C. in 1% solution of about 1500 to about 5500 mPas can be used to release the active ingredients in the sustained manner while exhibiting the mucoadhesion properties after insertion into the vaginal cavity as described above. In one embodiment, the hydrophilic matrix containing the aforementioned polymers (e.g., polyalkylene oxides) will surround the foregoing active pharmaceutical ingredients. The amount of the hydrophilic polymers such as the polyalkylene oxide in the composition will vary widely according to the molecular weight of the polymer. Generally, the % w/w of hydrophilic polymer for use in the pharmaceutical composition can range from about 1 to about 90% w/w, preferably about 2 to about 40% w/w and more preferably about 2 to about 20% w/w.

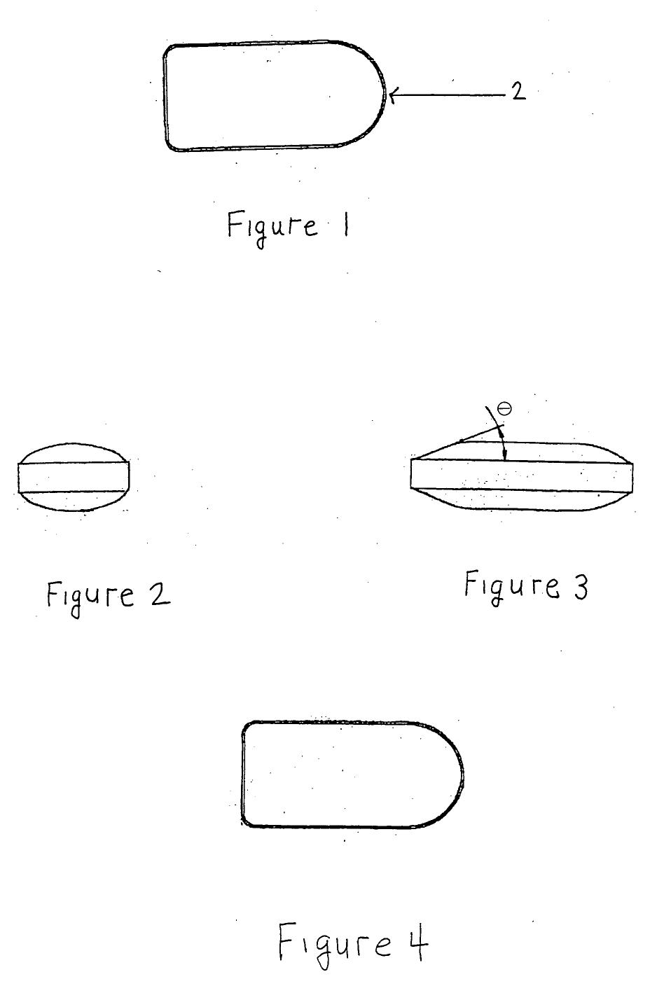

The pharmaceutical compositions of the present invention can be formulated in any manner. A preferred formulation for the pharmaceutical compositions herein are solid oral dosage forms such as, for example, tablets or hard capsules wherein the pharmacologically active ingredients are mixed with the hydrophilic matrix to form solid oral sustained release, mucoadhesive vaginal pharmaceutical composition according to procedures known in the art. The tablets or capsule can generally be of any size or shape for insertion into the vagina. In one embodiment, the pharmaceutical compositions are formulated into a tablet as a bullet shape as shown in FIGS. 1-4 and having the edges on one end of the tablet being beveled at an about 200 to about 30° angle and preferably about a 230 angle (see FIG. 3).

Tablets containing the compositions according to the present invention may be produced by any standard tabletting technique, e.g. by wet granulation, dry granulation or direct compression. For example, dry granulation procedures include mixing solid excipients, compacting the mixture in a compactor (e.g. a roller compactor) or double compression, milling the compacted mass, screening the milled granules, mixing with a lubricant and compressing the mixture into tablets. Direct compression procedures generally include mixing the solid excipients in one or more stages and compressing the uniform mixture into tablets.

The pharmaceutical compositions of the present invention containing the active pharmaceutical ingredients and hydrophilic matrix can contain one or more pharmaceutically acceptable excipients in accordance with known and established practice. The amount of the additional pharmaceutically acceptable excipients generally varies from about 10% to about 90% by weight, based on the total weight of the total composition.

The pharmaceutically acceptable excipients for use in the pharmaceutical compositions of the present invention include, but are not limited to, fillers, glidants, lubricants and the like and mixtures thereof that are typically used in the art for oral solid dosage forms.

Useful fillers may be inert fillers, either water soluble or water insoluble and selected from those typically used in the pharmaceutical art for oral solid dosage forms. Suitable fillers include, but are not limited to, calcium carbonate, dicalcium phosphate, tricalcium phosphate, microcrystalline cellulose, monosaccharide, disaccharides, polyhydric alcohols, sucrose, dextrose, lactose, fructose, mannitol, sorbitol, alone or mixtures thereof and the like or mixtures thereof. The amount of fillers can vary widely and will ordinarily range from about 1% to about 90% by weight, based on the total weight of the composition.

Useful glidants may be any glidant typically used in the pharmaceutical art for oral solid dosage forms. Examples include, but are not limited to, colloidal silicon dioxide, talc alone and the like or mixtures thereof. The amount of glidants can vary widely and will ordinarily range from about 0.1% to about 5.0% by weight, based on the total weight of the composition.

Useful lubricants can be any lubricant typically used in the pharmaceutical art for oral solid dosage forms. Example include, but are not limited to, stearate salts such as calcium stearate, magnesium stearate, zinc stearate and stearic acid, talc, hydrogenated vegetable oil, vegetable oil derivatives, silica, silicones, high molecular weight polyalkylene glycols and saturated fatty acids alone or mixtures and the like and mixtures thereof. The amount of lubricants can vary widely and will ordinarily range from about 0.1% to about 5.0% by weight, based on the total weight of the composition.

The pharmaceutical compositions of the present invention may contain other optional ingredients that are also typically used in pharmaceutical compositions such as, for example, coloring agents, preservatives, and the like and mixtures thereof. The amount of the optional ingredients can vary widely and will ordinarily range from about 0.1% to about 5.0% by weight, based on the total weight of the composition.

The pharmaceutical composition of the present invention can further contain a bulking agent. In one embodiment the bulking agent is a lactose monohydrate. Another preferred bulking agent is a corn starch. It is used widely in solid dosage formulations as a diluent and disintegrant.

The pharmaceutical composition of the present invention can further contain a binding agent. Binding agents of the present invention can be, for example, pyrrolidones such as a polyvinyl pyrrolidone (PVP) polymers and copolymers thereof, e.g., copolymers of polyvinylpyrrolidone with vinyl acetate, copolymers of polyvinyl with vinyl alcohol, copolymers of polyvinylpyrrolidone with vinyl chloride, copolymers of polyvinylpyrrolidone with vinyl fluoride, copolymers of polyvinylpyrrolidone with vinyl butyrate, copolymers of polyvinylpyrrolidone with vinyl laurate, copolymers of polyvinylpyrrolidone with vinyl stearate and the like and combinations thereof. The pyrrolidones for use herein can have a weight average molecular weight of from about 10,000 to about 3,000,000. A preferred binding agent is PVP K-25 available from ISP. Polyvinyl pyrrolidone is a synthetic polymer formed from linear 1-vinyl-2-pyrrolidone groups, the degree of polymerization of which results in polymers of various molecular weights. The PVP K25 polymer generally has about 30,000 units of linear 1-vinyl-2-pyrrolidone groups. In the present invention Povidone K-25 available from ISP can be used as a binder in wet-granulation processes. The amount of the binder can vary widely.

Another pharmaceutical excipient for use in the pharmaceutical compositions of the present invention is a submicroscopic fumed silica having a particle size of, for example, about 15 nm. It is generally a light, loose, bluish-white colored, odorless, tasteless, no gritty amorphous powder. As colloidal silicon dioxide has a small particle size and a large surface area give it desirable flow characteristics, and thus can be used as a glidant to improve the flow ability of the dried granules.

In another embodiment of the present invention, solid oral dosage forms of the pharmaceutical compositions herein are obtained by at least the following steps:
i) granulating the active pharmaceutical ingredient optionally along with at least one or more diluents and one or more binders;
ii) drying the granules, sizing and blending with the granules with one or more of the hydrophilic polymers and optionally a glidant;
iii) lubricating the blend of step (ii) with a lubricant; and
iv) tableting the mixture.

It is further within the scope of the invention to provide a kit containing the pharmaceutical compositions of the present invention and the apparatus necessary to carry out delivering the sustained release mucoadhesive, vaginal composition to the site of absorption or action in the vaginal cavity of a human female in the field. A complete kit would contain all of the equipment and consumables for conducting at least one test procedure, e.g., an applicator suitable for delivering the composition to the site in the vaginal cavity which are well known in the art. Such kits can also include, for example, other compounds and/or compositions (e.g., permeation enhancers, lubricants), and written instructions in a form prescribed by a governmental agency regulating the manufacture, use or sale of pharmaceuticals or biological products, which instructions can also reflects approval by the agency of manufacture, use or sale for human administration. The kit may be used, for example, in a hospital setting for administering the active pharmaceutical ingredient, e.g., oxitocin or petocin, by way of the applicator to a human female in the delivery room to induce labor. A partial test kit would include, at a minimum, the aforesaid pharmaceutical composition and the applicator.

The following example is provided to enable one skilled in the art to practice the invention and is merely illustrative of the invention. The examples should not be read as limiting the scope of the claims.

### EXAMPLE 1

### Preparation of a Sustained Release Mucoadhesive Vaginal Antifungal Pharmaceutical Tablet

The ingredients for use in this example are set forth below in Table I product was made by a granulation process and incorporating the active drug intragranular component.

TABLE 1

| Ingredients/Components | Qty ( mgs) | % w/w |
|---|---|---|
| Clotrimazole | 100.0 | 9.52 |
| PVP K-25 | 26.25 | 2.50 |
| Colloidal Silicon Dioxide | 5.25 | 0.50 |
| Corn Starch | 338.0 | 32.19 |
| Lactose Monohydrate | 535.25 | 50.98 |
| Polyox WSR 301* | 40.00 | 3.81 |
| Magnesium Stearate | 5.25 | 0.50 |
| Purified Water ** | — | |
| Total | 1050.0 | 100.0 |

*Polyox WSR 301 is a polyethylene oxide having a weight average molecular weight of 4,000,000
**Does not appear in final product.

First clotrimazole, corn starch, lactose monohydrate, and PVP K-25 were sifted through a # 30 mesh. Next, all of the remaining components listed in Table 1 together with the sifted clotrimazole, corn starch, lactose monohydrate, and PVP K-25 were blended together and then granulated using purified water as a binder to get the required consistency of the wet mass. The wet mass was then wet milled and the granules dried to get the require moisture content. The dried granules were then sized through a 30# mesh. The oversized granules were milled using a multimill with medium speed and knife forward and combined with the remaining granules. The granules were then lubricated using presifted Polyox WSR 301, colloidal silicon dioxide and magnesium stearate. The granules were ready for the compression.

It will be understood that various modifications may be made to the embodiments disclosed herein. Therefore the above description should not be construed as limiting, but merely as exemplifications of preferred embodiments. For example, the functions described above and implemented as the best mode for operating the present invention are for illustration purposes only. Other arrangements and methods may be implemented by those skilled in the art without departing from the scope and spirit of this invention. Moreover, those skilled in the art will envision other modifications within the scope and spirit of the claims appended hereto.

1. A sustained release, mucoadhesive vaginal pharmaceutical composition comprising (a) an effective amount of at least one active pharmaceutical ingredient and (b) a hydrophilic matrix having mucoadhesive properties and capable of providing a sustained release of the active pharmaceutical ingredient, the hydrophilic matrix comprising a hydrophilic polymer having a weight average molecular weight of at least about 100,000.

2. The pharmaceutical composition of claim 1, wherein the active pharmaceutical ingredient is selected from the group consisting of antifungal agents, prostaglandins, hormones, estrogens, pharmaceutically acceptable salts or esters thereof, isomers, derivatives thereof and combinations thereof.

3. The pharmaceutical composition of claim 2, wherein the antifungal agent is an azole-containing antifungal agent.

4. The pharmaceutical composition of claim 3, wherein the azole-containing antifungal agent is selected from the group consisting of imidazoles, triazoles, pharmaceutically acceptable salts or esters thereof, isomers, derivatives thereof and combinations thereof.

5. The pharmaceutical composition of claim 4, wherein the imidazole is selected from the group consisting of econazole, clotrimazole, metronidazole, tioconazole, fenticonazole, isoconazole, ketoconazole, sulconazole, bifonazole, omoconazole, azanidazole, butoconazole, oxiconazole and combinations thereof.

6. The pharmaceutical composition of claim 4, wherein the triazole is selected from the group consisting of fluconazole, terconazole, itraconazole and combinations thereof.

7. The pharmaceutical composition of claim 2, wherein the antifungal agent is clotrimazole.

8. The pharmaceutical composition of claim 1, wherein the hydrophilic polymer possesses a weight average molecular weight of at least about 500,000.

9. The pharmaceutical composition of claim 1, wherein the hydrophilic polymer possesses a weight average molecular weight of about 1,000,000 to about 10,000,000.

10. The pharmaceutical composition of claim 1, wherein the hydrophilic polymer possesses a weight average molecular weight of from about 2,000,000 to about 6,000,000.

11. The pharmaceutical composition of claim 1, wherein the hydrophilic polymer is a polyalkylene oxide.

12. The pharmaceutical composition of claim 11, wherein the polyalkylene oxide is a polyethylene oxide.

13. The pharmaceutical composition of claim 11, wherein the polyalkylene oxide possesses a weight average molecular weight of at least about 500,000.

14. The pharmaceutical composition of claim 11, wherein the polyalkylene oxide possesses a weight average molecular weight of about 1,000,000 to about 10,000,000.

15. The pharmaceutical composition of claim 11, wherein the polyalkylene oxide possesses a weight average molecular weight of from about 2,000,000 to about 6,000,000.

16. The pharmaceutical composition of claim 1, further comprising one or more pharmaceutically acceptable excipients.

17. The pharmaceutical composition of claim 1, wherein the polymer is present in the composition from about 1 to about 90% w/w.

18. The pharmaceutical composition of claim 1, wherein the polymer is present in the composition from about 1 to about 40% w/w.

19. The pharmaceutical composition of claim 1, wherein the polymer is present in the composition from about 2 to about 20% w/w.

20. The pharmaceutical composition of claim 1, which is a solid oral dosage form.

21. The pharmaceutical composition of claim 1, wherein the solid oral dosage form is a tablet.

22. A process for preparing a sustained release mucoadhesive vaginal pharmaceutical tablet comprising:
(a) granulating at least one active pharmaceutical ingredient;
(b) blending the granules with a hydrophilic polymer having a weight average molecular weight of at least about 100,000 to form a mixture; and,
(c) tableting the mixture thereby obtained.

23. The process of claim 22, wherein the hydrophilic polymer possesses a weight average molecular weight of at least about 500,000.

24. The process of claim 22, wherein the hydrophilic polymer possesses a weight average molecular weight of about 1,000,000 to about 10,000,000.

25. The process of claim 22, wherein the hydrophilic polymer possesses a weight average molecular weight of from about 2,000,000 to about 6,000,000.

26. The process of claim 22, wherein the hydrophilic polymer is a polyalkylene oxide.

27. The process of claim 26, wherein the polyalkylene oxide is a polyethylene oxide.

28. The process of claim 22, wherein the step of granulating further comprises adding a diluent.

29. The process of claim 22, wherein the step of granulating further comprises adding a binder.

30. The process of claim 29, wherein the binder is polyvinylpyrollidone.

31. The process of claim 22, wherein the step of blending comprises adding a glidant.

32. The process of claim 22, further comprising the step of lubricating the blend of step (b) with a lubricant.

33. The process of claim 32, wherein the lubricant is magnesium stearate.

34. A method for treating a vaginal condition in a human female is provided comprising the step of administering into the vaginal cavity of the human female a solid oral sustained release, mucoadhesive vaginal pharmaceutical composition comprising an effective amount of at least one active pharmaceutical ingredient and a hydrophilic matrix having mucoadhesive properties and capable of providing a sustained release of the active pharmaceutical ingredient, the hydrophilic matrix comprising a hydrophilic polymer having a weight average molecular weight of at least about 100,000.

35. The method of claim 34, wherein the active pharmaceutical ingredient is selected from the group consisting of antifungal agents, prostaglandins, hormones, estrogens, pharmaceutically acceptable salts or esters thereof, isomers, derivatives thereof and combinations thereof.

36. The method of claim 35, wherein the antifungal agent is an azole-containing antifungal agent.

37. The method of claim 36, wherein the azole-containing antifungal agent is selected from the group consisting of imidazoles, triazoles, pharmaceutically acceptable salts or esters thereof, isomers, derivatives thereof and combinations thereof.

38. The method of claim 34, wherein the active pharmaceutical ingredient is clotrimazole.

39. The method of claim 34, wherein the hydrophilic polymer possesses a weight average molecular weight of at least about 500,000.

40. The method of claim 34, wherein the hydrophilic polymer possesses a weight average molecular weight of about 1,000,000 to about 10,000,000.

41. The method of claim 34, wherein the hydrophilic polymer possesses a weight average molecular weight of from about 2,000,000 to about 6,000,000.

42. The method of claim 34, wherein the hydrophilic polymer is a polyalkylene oxide.

43. The method of claim 42, wherein the polyalkylene oxide is a polyethylene oxide.

44. The method of claim 42, wherein the polyalkylene oxide possesses a weight average molecular weight of at least about 500,000.

45. The method of claim 42, wherein the polyalkylene oxide possesses a weight average molecular weight of about 1,000,000 to about 10,000,000.

46. The method of claim 42, wherein the polyalkylene oxide possesses a weight average molecular weight of from about 2,000,000 to about 6,000,000.

47. The method of claim 34, further comprising one or more pharmaceutically acceptable excipients.

48. The method of claim 34, wherein the polymer is present in the composition from about 1 to about 90% w/w.

49. The method of claim 34, wherein the polymer is present in the composition from about 1 to about 40% w/w.

50. The method of claim 34, wherein the polymer is present in the composition from about 2 to about 20% w/w.

51. The method of claim 34, wherein the solid oral pharmaceutical composition is a tablet.

52. A pharmaceutical kit comprising the sustained release mucoadhesive vaginal pharmaceutical composition of claim 1 and an applicator.

53. The pharmaceutical kit of claim 52, wherein the sustained release mucoadhesive vaginal pharmaceutical composition is a solid oral dosage form.

54. The pharmaceutical kit of claim 52, wherein the solid oral dosage form is a tablet.
